(19) United States
(12) Patent Application Publication
(10) Pub. No.: US 2015/0329509 A1
(43) Pub. Date: Nov. 19, 2015

(54) PROCESSES FOR THE PREPARATION OF ENAMINES

(71) Applicant: Dow AgroSciences LLC, Indianapolis, IN (US)

(72) Inventors: Douglas C. Bland, Midland, MI (US); Todd William Toyzan, Freeland, MI (US)

(73) Assignee: DOW AGROSCIENCES LLC, Indianapolis, IN (US)

(21) Appl. No.: 14/808,017

(22) Filed: Jul. 24, 2015

Related U.S. Application Data
(63) Continuation of application No. 14/172,516, filed on Feb. 4, 2014.
Application No. 14/172,516 is a continuation of application No. 13/303,195, filed on Nov. 23, 2011, now abandoned.
(60) Provisional application No. 61/419,300, filed on Dec. 3, 2010.

(51) Int. Cl. C07D 295/084

(52) U.S. Cl. CPC C07D 295/084

(57) ABSTRACT

The invention disclosed in this document is related to the field of processes for the preparation of enamines

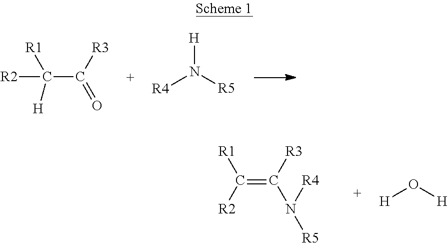

wherein R1, R2, R3, R4, R5, and further information are disclosed herein.

### CROSS-REFERENCE TO RELATED APPLICATIONS

This Application is a continuation of, and claims the benefit of, U.S. non-provisional application Ser. No. 14/172,516, which was filed on Feb. 4, 2014, and which is a continuation of, and claims the benefit of, U.S. non-provisional application Ser. No. 13/303,195, which was filed on Nov. 23, 2011, which claims priority from, and benefit of, U.S. provisional application 61/419,300, which was filed on Dec. 3, 2010. The entire content of these applications are hereby incorporated by reference into this Application.

### FIELD OF THE INVENTION

The invention disclosed in this document is related to the field of processes for the preparation of enamines.

### BACKGROUND OF THE INVENTION

Enamines are very useful molecules. They have been used in a wide variety of reactions such as, for example, electrophilic substitution and addition, oxidation and reduction, and cycloaddition (J. Kang, Y. R. Cho, and J. H. Lee, Bull. Korean Chem Soc. Vol. 13, No. 2, 1992).

An early method for preparing enamines involved the condensation of aldehydes and ketones with secondary amines (C. Mannich and H. Davidsen, Ber., 69, 2106 (1936). Mannich and Davidsen discovered that the condensation reaction of an aldehyde with a secondary amine could be conducted at temperatures near 0° C. in the presence of potassium carbonate (K₂CO₃), but however, the condensation reaction of a ketone with a secondary amine required calcium oxide (CaO) and elevated temperatures. Later, Herr and Heyl discovered that this type of condensation reaction could be improved by removing water (H₂O) during an azeotropic distillation with benzene (M. E. Herr and F. W. Heyl, J. Am. Chem. Soc., 74, 3627 (1952); F. W. Heyl and M. E. Herr, J. Am. Chem. Soc., 75, 1918 (1953); M. E. Herr and F. W. Heyl, J. Am. Chem. Soc., 75, 5927 (1953); F. W. Heyl and M. E. Herr, J. Am. Chem. Soc., 77, 488 (1955)). Since these publications a number of modifications have been disclosed. Usually, these modifications are based on using dehydration reagents such as K₂CO₃, CaO, p-toluenesulfonic acid (TsOH), boron trifluoride diethyl etherate (BF₃—OEt₂), acetic acid (AcOH), magnesium sulfate (MgSO₄), calcium hydride (CaH₂), titanium tetrachloride (TiCl₄), and molecular sieves (see J. Kang above). Other modifications deal with chemically converting water to something else during the condensation reaction (see J. Kang above). An extensive summary of the vast number of methods to prepare enamines is discussed in “ENAMINES, Synthesis, Structure, and Reactions,” 2^{nd} Edition, Edited by A. G. Cook, Chap. 2, (1988). Specific examples of processes to prepare enamines can be found in the following:
U.S. Pat. No. 3,074,940 which discloses that certain aldehydes form azeotropes with water which can be used to remove the reaction water formed during certain enamine condensation reactions;
U.S. Pat. No. 3,530,120 which discloses conducting certain enamine condensation reactions in an inert atmosphere with certain arsine molecules;
U.S. Pat. No. 5,247,091 which discloses conducting certain enamine condensation reactions in an aqueous media;
S. Kaiser, S. P. Smidt, and A. Pfaltz, Angew. Int. Ed. 2006, 45, 5194-5197—See Supporting information pages 10-11; and
WO 2009/007460 A2, see page 13, example 1.a.

Enamines such as 1-(3-thiobut-1-enyl)pyrrolidine are useful intermediates for the preparation of certain new insecticides (see, for example, U.S. Patent Publications 2005/0228027 and 2007/0203191). Current known processes to make such thioenamines are not efficient in producing such enamines due to a variety of reasons—there are problems in preventing thermal degradation of the thioenamine, and while using potassium carbonate is an effective desiccant, it is problematic to filter such desiccant during larger than lab-scale production. Thus, a process is needed to remove water during these types of condensation reactions without using solid desiccants, or using temperature conditions that promote the thermal degradation of such enamines.

### DETAILED DESCRIPTION OF THE INVENTION

In general, the processes disclosed in this document can be illustrated as in Scheme 1.

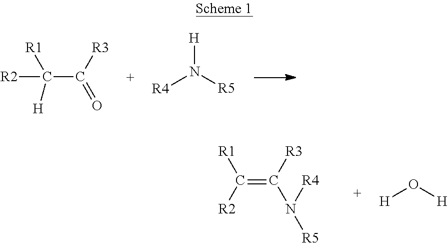

In general, the invention is a process comprising:

(A) reacting, in a reaction zone that comprises a solvent, an amine and a carbonyl to produce an enamine and H₂O
(1) wherein said amine has the following formula

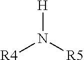

wherein R4 and R5 are each independently selected from C₁-C₈ alkyl, C₃-C₈ cycloalkyl, C₂-C₈ alkoxyalkyl, C₇-C₁₂ arylalkyl, C₂-C₈ alkylaminoalkyl, aryl, and heteroaryl, or R4 and R5 taken together with N represent a 5- or 6-membered saturated or unsaturated ring; and
(2) wherein said carbonyl (i.e. an aldehyde or a ketone) has the following formula

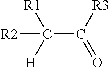

(a) wherein R1 and R2 is each independently selected from C₁-C₈ alkyl, C₃-C₈ cycloalkyl, C₂-C₈ alkoxyalkyl, C₇-C₁₂ arylalkyl, C₂-C₈ alkylaminoalkyl, aryl, and heteroaryl, each of which is independently substituted with one or more S—R6 wherein each R6 is independently selected from C₁-C₈ alkyl, C₃-C₈ cycloalkyl, C₂-C₈ alkoxyalkyl, C₇-C₁₂ arylalkyl, C₂-C₈ alkylaminoalkyl, aryl, and heteroaryl, and
(b) wherein R3 is selected from H, C₁-C₈ alkyl, C₃-C₈ cycloalkyl, C₂-C₈ alkoxyalkyl, C₇-C₁₂ arylalkyl, C₂-C₈ alkylaminoalkyl, aryl, and heteroaryl;
(3) wherein said reacting, in said reaction zone, is conducted under distillation conditions comprising
   (a) a pressure from about 100 Pascals (Pa) to about 120,000 Pa, and
   (b) a temperature below about, but preferably below, the thermal decomposition temperature of said enamine during said reacting; and
(4) wherein said solvent initially comprises a non-polar-high-boiling-point liquid, a polar-high-boiling-point-liquid, and then further comprises H₂O produced from the condensation of said amine and said carbonyl to produce said enamine; and

(B) removing a vapor phase from said reaction zone wherein said vapor phase comprises H₂O.

Approximately equimolar quantities of said amine and said carbonyl can be used in the process, although excesses of one or the other may be employed. The molar ratio of amine to carbonyl can be from about 0.9 to about 1.2, however, a slight molar excess of amine to carbonyl is preferred, such as, for example, a molar ratio greater than 1 but less than about 1.1.

The reaction is conducted in the presence of a solvent that initially comprises:

(1) non-polar-high-boiling-point-liquid such as, hydrocarbon liquids, most preferably aromatic hydrocarbon liquids such as, for example, benzene, toluene, or xylene. Currently, toluene is a preferred liquid;

(2) polar-high-boiling-point-liquid such as, acetonitrile, ethanol; and then

(3) further comprises H₂O produced from the condensation of said amine and said carbonyl to produce said enamine.

In another embodiment of this invention said reacting is conducted under distillation conditions comprising a pressure from about 1000 Pa to about 60,000 Pa and a temperature from about 10° C. to about 80° C.

In another embodiment of this invention said reacting is conducted under distillation conditions comprising a pressure from about 2500 Pa to about 30,000 Pa and a temperature from about 20° C. to about 70° C.

In another embodiment of this invention said reacting is conducted under distillation conditions comprising a pressure from about 5000 Pa to about 15,000 Pa and a temperature from about 25° C. to about 65° C. In another embodiment of this invention when producing 1-(3-methylsulfanyl-but-1-enyl)-pyrrolidine a temperature below about the thermal decomposition temperature of 1-(3-methylsulfanyl-but-1-enyl)-pyrrolidine during said reacting is preferred.

It is preferred in such processes that the H₂O be removed under azeotropic conditions. It is also preferred if no desiccants be used to remove H₂O.

In another embodiment of this invention, R1 and R2 are independently C₁-C₈ alkyl, C₃-C₈ cycloalkyl, each of which is independently substituted with one or more S—R6 wherein each R6 is independently selected from C₁-C₈ alkyl.

In another embodiment of this invention, R3 is H.

In another embodiment of this invention, wherein R4 and R5 are each independently selected from C₁-C₈ alkyl and C₃-C₈ cycloalkyl. In another embodiment of this invention R4 and R5 taken together with N represent a 5- or 6-membered saturated or unsaturated ring.

In another embodiment of this invention, said amine is pyrrolidine and said carbonyl is 3-methylsulfanyl-butyraldehyde. In another embodiment of this invention, said enamine is 1-(3-methylsulfanyl-but-1-enyl)-pyrrolidine.

### EXAMPLES

The examples are for illustration purposes and are not to be construed as limiting the invention disclosed in this document to only the embodiments disclosed in these examples.

### Comparative Example

### Preparation of 1-(3-methylthiobut-1-enyl)pyrrolidine

A three-neck 250 mL round bottom flask equipped with a short path distillation head was connected to a receiver flask containing a dry-ice acetone condenser. To this reaction vessel was charged 19.8 g (0.28 mol) of pyrrolidine followed by 70 mL of toluene. The mixture was cooled in an ice-water bath until the internal reaction pot temperature was about 3° C. Then vacuum (about 3300 Pa) was applied to the system and then 94.4 g (0.14 mol) of 3-methylthiobutanal as a 17.5 wt % solution in toluene was continuously added to the reaction mixture via syringe over a one hour period. The internal reaction temperature rose from 3° C. up to 18° C. during addition of the aldehyde solution. Distillate was also collected during aldehyde addition. Upon completing addition of the 3-methylthiobutanal solution, the distillation was continued for an additional 50 minutes (min) until the internal pot temperature reached 26° C. At this time, the vacuum was adjusted to about 2400 Pa and the distillation was continued for an additional 2.0 min until the internal pot temperature reached 24° C. The distillation was stopped and the reaction vessel was padded with nitrogen. The reactive distillation bottoms were isolated to give 74.91 g of 1-(3-methylthiobut-1-enyl)pyrrolidine was a 28 wt % yellow solution in toluene. Proton (¹H) NMR spectroscopic assay of the solution mixture (using benzyl acetate as the internal standard) indicated a 84% in-pot yield.

### Example 1

### Preparation of 1-(3-methylthiobut-1-enyl)pyrrolidine

To a 3-Liter three-neck round bottom flask equipped with mechanical stirring, short path distillation head, and nitrogen padding was charged with 61 g (0.86 mol) of pyrrolidine followed by 100 mL of toluene and 200 mL of acetonitrile (33% toluene in acetonitrile v/v). The mixture was cooled in an ice-water bath and then 558 g (0.78 mol) of a 16.5 wt % 3-methylthiobutanal in toluene solution was continuously added via additional funnel over a 130 min period. The internal reaction temperature was maintained below 7° C. during the aldehyde addition. The ice-water bath was removed and a pressure of about 6600 Pa was applied to the system. The reaction mixture was heated up to 15° C. (pot temperature) at which time distillate began to be collected overhead. The internal reaction temperature was heated until the pot temperature reached 33° C. Total time for the distillation was about 1 h. The reaction mixture was padded with nitrogen and then cooled down to ambient temperature. A total of 282.4 g of overhead distillate was collected. The reaction distillation bottoms were collected to give a about 25.0 wt % 1-(3-methylthiobut-1-enyl)pyrrolidine in toluene solution (yield was approximated to be 89% based on ¹H NMR spectroscopy using benzyl acetate as an internal standard).

What is claimed is:

1. A process comprising:
(A) reacting, in a reaction zone that comprises a solvent, an amine, and a carbonyl to produce an enamine and H₂O
(1) wherein said amine has the following formula 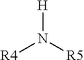
wherein R4 and R5 are each independently selected from C₁-C₈ alkyl, C₃-C₈ cycloalkyl, C₂-C₈ alkoxyalkyl, C₇-C₁₂ arylalkyl, C₂-C₈ alkylaminoalkyl, aryl, and heteroaryl, or R4 and R5 taken together with N represent a 5- or 6-membered saturated or unsaturated ring; and
(2) wherein said carbonyl is an aldehyde or a ketone and has the following formula 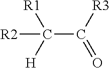
(a) wherein R1 and R2 is each independently selected from C₁-C₈ alkyl, C₃-C₈ cycloalkyl, C₂-C₈ alkoxyalkyl, C₇-C₁₂ arylalkyl, C₂-C₈ alkylaminoalkyl, aryl, and heteroaryl, each of which is independently substituted with one or more S—R6 wherein each R6 is independently selected from C₁-C₈ alkyl, C₃-C₈ cycloalkyl, C₂-C₈ alkoxyalkyl, C₇-C₁₂ arylalkyl, C₂-C₈ alkylaminoalkyl, aryl, and heteroaryl, and
(b) wherein R3 is selected from H, C₁-C₈ alkyl, C₃-C₈ cycloalkyl, C₂-C₈ alkoxyalkyl, C₇-C₁₂ arylalkyl, C₂-C₈ alkylaminoalkyl, aryl, and heteroaryl;
(3) wherein said reacting, in said reaction zone, is conducted under distillation conditions comprising
(a) a pressure from about 100 Pa to about 120,000 Pa, and
(b) a temperature below about, but preferably below, the thermal decomposition temperature of said enamine during said reacting; and
(4) wherein said solvent initially comprises a non-polar-high-boiling-point liquid, a polar-high-boiling-point-liquid, and then further comprises H₂O produced from the condensation of said amine and said carbonyl to produce said enamine; and
(B) removing a vapor phase from said reaction zone wherein said vapor phase comprises H₂O.

2. A process according to claim 1 wherein approximately equimolar quantities of said amine and said carbonyl can be used in the process.

3. A process according to claim 1 wherein the molar ratio of amine to carbonyl is from about 0.9 to about 1.2.

4. A process according to claim 1 wherein molar ratio of amine to carbonyl is greater than 1 but less than about 1.1.

5. A process according to claim 1 wherein the reaction is conducted in the presence of a solvent that initially comprises said non-polar-high-boiling-point-liquid where said liquid is benzene.

6. A process according to claim 1 wherein the reaction is conducted in the presence of a solvent that initially comprises said non-polar-high-boiling-point-liquid where said liquid is toluene.

7. A process according to claim 1 wherein the reaction is conducted in the presence of a solvent that initially comprises said non-polar-high-boiling-point-liquid where said liquid is xylene.

8. A process according to claim 1 wherein the reaction is conducted in the presence of a solvent that initially comprises said polar-high-boiling-point-liquid where said liquid is acetonitrile.

10. A process according to claim 1 wherein the reaction is conducted in the presence of a solvent that initially comprises said polar-high-boiling-point-liquid where said liquid is ethanol.

11. A process according to claim 1 wherein said reacting is conducted under distillation conditions comprising a pressure from about 1000 Pa to about 60,000 Pa and a temperature from about 10° C. to about 80° C.

12. A process according to claim 1 wherein said reacting is conducted under distillation conditions comprising a pressure from about 2500 Pa to about 30,000 Pa and a temperature from about 20° C. to about 70° C.

13. A process according to claim 1 wherein said reacting is conducted under distillation conditions comprising a pressure from about 5000 Pa to about 15,000 Pa and a temperature from about 25° C. to about 65° C.

14. A process according to claim 1 wherein 1-(3-methylsulfanyl-but-1-enyl)-pyrrolidine is produced at a temperature below about the thermal decomposition temperature of 1-(3-methylsulfanyl-but-1-enyl)-pyrrolidine.

15. A process according to claim 1 wherein said H₂O is removed under azeotropic conditions.

16. A process according claim 1 wherein no desiccants are used to remove H₂O.

17. A process according to claim 1 wherein said R1 and R2 are independently C₁-C₈ alkyl, C₃-C₈ cycloalkyl, each of which is independently substituted with one or more S—R6 wherein each R6 is independently selected from C₁-C₈ alkyl.

18. A process according to claim 1 wherein R3 is H.

19. A process according to claim 1 wherein R4 and R5 are each independently selected from C₁-C₈ alkyl and C₃-C₈ cycloalkyl.

20. A process according to claim 1 wherein R4 and R5 taken together with N represent a 5- or 6-membered saturated or unsaturated ring.

21. A process according to claim 1 wherein said amine is pyrrolidine and said carbonyl is 3-methylsulfanyl-butyraldehyde.

22. A process according to claim 1 wherein said enamine is 1-(3-methylsulfanyl-but-1-enyl)-pyrrolidine.

23. A process to produce 1-(3-methylsulfanyl-but-1-enyl)-pyrrolidine said process consisting essentially of:
(A) reacting, in a reaction zone that comprises solvents, pyrrolidine and 3-methylsulfanyl-butyraldehyde to produce 1-(3-methylsulfanyl-but-1-enyl)-pyrrolidine and H₂O, wherein said reacting, in said reaction zone, is conducted under azeotropic distillation conditions consisting essentially of
(1) a pressure from 5000 Pa to 15,000 Pa, and
(2) a temperature from 25° C. to 65° C.; and
wherein said solvents are initially toluene and acetonitrile, and then H₂O, where said H₂O is produced from the condensation of said pyrrolidine and said 3-methylsulfanyl-butyraldehyde to produce said 1-(3-methylsulfanyl-but-1-enyl)-pyrrolidine, thereby forming a ternary azeotrope of toluene, acetonitrile, and H₂O; and
(B) removing a vapor phase from said reaction zone wherein said vapor phase consists essentially of comprises H₂O;
wherein said process no desiccants are used to remove H₂O;
wherein said process said molar ratio of pyrrolidine to 3-methylsulfanyl-butyraldehyde is greater than 1 but less than 1.1.
